(19) United States
(12) Patent Application Publication
(10) Pub. No.: US 2019/0314297 A1
(43) Pub. Date: Oct. 17, 2019

(54) COMBINATION THERAPY OF CBD AND COPAXONE

(71) Applicant: TO PHARMACEUTICALS LLC, New York, NY (US)

(72) Inventor: Ruth GALLILY, Jerusalem (IL)

(73) Assignee: TO PHARMACEUTICALS LLC, New York, NY (US)

(21) Appl. No.: 16/346,906

(22) PCT Filed: Nov. 2, 2017
(86) PCT No.: PCT/IL2017/051200

§ 371 Date: May 2, 2019

Related U.S. Application Data
(60) Provisional application No. 62/416,265, filed on Nov. 2, 2016.

(51) Int. Cl. A61K 31/05; A61K 38/02; A61K 9/00; A61P 25/28

(52) U.S. Cl. CPC A61K 31/05; A61P 25/28; A61K 9/0019; A61K 38/02

(57) ABSTRACT

Provided are concerns and methods of using glatiramer acetate (GA) and cannabidiol (CBD), for treating, preventing, ameliorating or delaying MS, and side effects associated with MS treatment.

### TECHNOLOGICAL FIELD

The present disclosure relates to a combination therapy for treating autoimmune diseases and specifically for treating multiple sclerosis.

### BACKGROUND

Glatiramer acetate (GA) also known as Copaxone (Teva pharmaceuticals) is used in reducing the frequency of relapses in patients with relapsing-remitting multiple sclerosis (RRMS), and in patients who have experienced a first clinical episode of Multiple Sclerosis (MS).

Cannabidiol (CBD) is one of at least 113 active cannabinoids identified in cannabis. It is one of the major phytocannabinoids, accounting for up to 40% of plant's extract. CBD is considered to have a wide scope of potential medical applications—due to clinical reports showing the lack of side effects, particularly a lack of psychoactivity (as is typically associated with Δ9-THC), and non-interference with several psychomotor learning and psychological functions.

Compositions comprising GA and cannabidiol (CBD) for use in the treatment of MS via nasal administration were described in [1].

U.S. Pat. No. 6,410,588 [2] discloses the use of CBD for the treatment of inflammatory conditions such as Rheumatoid Arthritis (RA), MS and inflammatory bowel disease (IBD).

### BACKGROUND ART

[1] WO 2008/120207
[2] U.S. Pat. No. 6,410,588
[3] Gallily R et al., Overcoming the Bell Shaped Dose-Response of Cannobidiol by using Cannabis Extract enriched in Cannabidiol, Pharmacology & Pharmacy 2015; 6, 75-85.

### SUMMARY OF THE INVENTION

The present disclosure is generally based on the findings that a combination therapy comprising glatiramer acetate (GA) and cannabidiol (CBD) is effective in treating multiple sclerosis (MS) and also effective in reducing side effects (or adverse reactions) associated with administration of GA, e.g., via injection, subcutaneous (s.c.) injection in particular.

Glatiramer acetate (GA, Copaxone, copolymer 1), for injection, is an approved drug for chronic or relapsing-remitting MS. The clinical and immunological effects of GA were extensively studied in experimental autoimmune encephalomyelitis (EAE), the experimental animal model for MS, and in human clinical studies. The commercial Copaxone is intended for subcutaneous use only (it is not administered intravenously) with a dosing schedule dependent on product strength. The most common adverse reactions of Copaxone administration, leading in at least 5% of patients to discontinuation of treatment, include, inter alia, injection site reactions, urticaria, vasodilatation, rash, dyspnea, hypersensitivity and chest pain.

Specifically, the inventor of the technology disclosed herein has found that administration of a preparation comprising CBD, typically in purified form or in a form consisting CBD, potentiates the effect of GA on the symptoms of MS as revealed in an animal model of MS. In particular, CBD was found effective for the reduction of skin lesions associated with s.c. administration of GA (also referred to as a local injection site reaction).

While certain combinations of GA and CBD for nasal administration have been previously proposed for the treatment of MS [1], they were found unsuitable for prolonged treatment of chronic MS due to irritation of nasal mucosa, and are considered more useful for immediate alleviation of acute MS.

More specifically, as demonstrated herein, a combination therapy using a subcutaneous (s.c.) administration of GA and an intraperitoneal (i.p) administration of CBD was more effective in treatment of MS compared to GA or CBD alone (see FIG. 4). The combination therapy according to the invention was not only effective for the reduction of pathological manifestations of MS, but was further effective for the reduction of side effects associated with GA s.c. injection in particular.

Thus, in one of its main aspects the invention provides a method for treating, preventing, or ameliorating MS, or delaying MS onset, in a subject in need thereof (e.g., a subject suffering therefrom or who is predicted to suffer from the disease or who has been diagnosed to potentially develop the disease), the method comprising administrating to the subject a combination therapy comprising a therapeutically effective amount of GA and a therapeutically effective amount of CBD.

It should be appreciated that the presently proposed combination therapy is applicable, in some embodiments, when GA is administered by injection, s.c. injection in particular.

An important attribute of the presently conceived combination therapy is that it is effective for treating, preventing, ameliorating or delaying the onset of MS or the recurrence of MS episodes compared to each one of its components, GA or CBD, alone.

In yet another aspect, the present invention provides a method for reducing, inhibiting, attenuating or eliminating at least one side effect associated with administration of GA to a subject, said method comprising administering an effective amount of CBD to the subject, such that the effective amount is sufficient to reduce at least one said side effect of GA. In instances wherein the GA is administered by s.c. injection, the at least one side effect can be associated with s.c. injection of GA.

According to the invention, the subject to be treated with the presently proposed combination therapy is one which has been found or determined to possibly benefit from the treatment with GA, and for whom a therapeutic protocol has been tailored or proposed, specifying effective amounts of the GA and the CBD, such that the amount of one component is adjusted to or determined based on the amount of the other component or to any one other protocol parameter which may, inter alia, depend on subject health and personal factors as well as on time of administration, sequence and administration regimen.

More specifically, in certain cases the subject to be treated is one who, at the time of assessment, is treated with an injected GA, and the amount and frequency of administration of the CBD is determined in consideration of the concurrent GA treatment. In other cases, the subject can be one who is predisposed, suspected or known to suffer from injected GA-related side effects, whereby the administration of CBD, as defined herein, assists in reducing or diminishing such side effects.

Yet in other cases the CBD can be administered prior to the administration of GA. In some embodiments, the CBD is administered immediately after administration of GA. In other embodiments, the two are administered simultaneously either in separate dosage forms or as a single mixed composition of matter comprising GA and CBD in a carrier suitable for injection, preferably for s.c. injection.

As described herein, it was found that a combination therapy comprising CBD and GA, e.g., s.c injected, has profound beneficial effects, both in augmenting therapeutic effects and in reducing side effects, associated with (injectable) GA. It should be understood that reference to administration of GA with CBD in ‘combination’ or ‘together’ refers to a treatment schedule involving more than one type of therapy. According to the present disclosure, a combination therapy denotes a regimen involving administration of at least two substances in a single treatment cycle. As may be appreciated, the combination therapy requires an assessment of various parameters, inter alia, treatment schedule, predetermined ratio of the CBD and GA (dosing), number of treatment cycles, and others. Assessment of the combination therapy may be carried out before onset of treatment. For example, the CBD may be administered during the period at which a patient is treated with GA, at any time before administration of GA or at any time after administration of GA, provided that the later administration is at a time sufficient to yield an effective combination therapy.

The CBD and GA described herein can be administered and dosed by methods of the invention, in accordance with good medical practice, such as systemically, for example by parenteral, e.g. intravenous, intraperitoneal or intramuscular injection. In another example, the CBD and GA can be introduced to a site by any suitable route including intravenous, subcutaneous, transcutaneous, topical, intramuscular, intraarticular, subconjunctival, or mucosal, oral, or intraocular administration. The administration of the two components may be by the same or different modes of administration and at the same or different frequency, i.e. at the same or different time points.

The present disclosure also provides a pharmaceutical composition comprising GA and CBD, one of which or each in a carrier. In such compositions GA and CBD may be present in the same composition, adapted for parenteral, oral, transdermal administration. In some cases such compositions are adapted for s.c. injection, including a carrier adapted for s.c. injection. The pharmaceutical compositions are applied for treating, preventing, ameliorating or delaying the onset of MS, in a subject suffering therefrom or who is predicted to suffer from the disease or who has been diagnosed to potentially develop the disease.

In some cases, as demonstrated in the present Examples, the GA and the CBD can be administered simultaneously or in succession, as separate compositions adapted for either parenteral, oral, transdermal (i.e., s.c.) administration. In certain embodiments, both the GA and the CBD can be adapted for an administration by s.c. injection.

As described herein, the presently proposed combination therapy can be relevant to administration of injectable GA, which has related side effects such as skin lesions or site reactions, or pain (Copaxone prescribing information by Teva Pharmaceuticals USA, Inc, North Wales, 2009).

The invention further provides use of GA for the preparation of a composition to be administered in combination with CBD.

Still further, the invention provides use of CBD for the preparation of a composition to be administered in combination with GA.

In yet another aspect, the invention provides a kit comprising, in separate reservoirs, an effective amount of GA and an effective amount of CBD, the kit further comprising instructions for using the effective amount of GA and effective amount of CBD in a combination therapy.

Each one of the components, the GA and the CBD, are act better in combination than alone, articulated herein by the terms ‘more effective’ or ‘potentiated’ effects. Such potentiated effects can have clinical manifestations of increased therapeutic effects or reduced side effects associated with each one of the components, and also in terms of therapeutically effective doses of the components when administered alone.

Thus, in yet another aspect, the invention provides an effective amount of injected GA, preferably s.c. injected GA, for use in a method of administering to a subject an effective amount of CBD, wherein the effective amount of GA is selected to potentiate at least one effect associated with CBD.

In a further aspect the invention provides an effective amount of CBD for use in a method of administering to a subject an effective amount of injected GA, preferably s.c. injected GA, wherein the effective amount of CBD is selected to potentiate at least one effect associated with GA.

The invention also provides an effective amount of CBD for use in a method of administering to a subject an effective amount of injected GA, wherein the effective amount of the CBD is selected to reduce or diminish at least one side effect associated with the administration to a subject of injected GA.

As noted herein, the present disclosure refers to MS (formerly known as disseminated sclerosis or encephalomyelitis disseminate), a chronic, inflammatory, demyelinating disease that affects the central nervous system (CNS). The disease onset usually occurs in young adults with a prevalence between 2 and 150 per 100,000 depending on specific population and country. MS is characterized by presence of multiple (at least two) neurological attacks affecting the CNS, manifested in the form of demyelinating lesions on brain magnetic resonance imaging (MRI). MS takes several forms, with new symptoms occurring either in discrete episodes (relapsing forms) or slowly accumulating over time (progressive forms). Most people are first diagnosed with relapsing-remitting MS (RRMS), and develop secondary-progressive MS (SPMS) after a number of years. Between episodes or attacks, symptoms can withdraw completely, although permanent neurological problems often persist, especially in advanced disease. RRMS occurs in about in 85% percent of the patients and SPMS in about 15%. It should be appreciated that the protocols, compositions and kits of the invention are applicable for the treatment of both MS forms, RRMS and SPMS, in children, young adults and adult subjects.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows results of an experiment in EAE model in mice treated by control (vehicle) and 5 mg/kg of CBD injected ip.

FIG. 2 shows another experiment using EAE model in mice treated by control (vehicle) and 5 mg/kg of CBD injected ip.

FIG. 3 shows EAE mice treated with and 5 mg/kg of CBD injected ip and GA (Copaxone) (1 mg/mice injected s.c.)

FIG. 4 shows EAE mice treated with control (vehicle), CBD alone (5 mg/kg injected i.p), GA(Copaxone)(1 mg/mice injected s.c) and a combination of CBD and Copaxone.

### DETAILED DESCRIPTION OF EMBODIMENTS

Before describing the invention it should be noted that it is not limited to herein described methods and experimental conditions, as well as the terminology used herein for describing particular embodiments is not intended to be limiting. Unless defined otherwise, all technical and scientific terms used herein have the meaning as commonly understood by one of ordinary skill in the art to which this invention pertains. Although any methods and materials similar or equivalent to those described herein can be used in the practice or testing of the invention, particular methods and materials are now described.

One aspect of the invention is to provide a method of treating, preventing, ameliorating MS or delaying MS onset in a subject in need thereof (e.g., a subject suffering therefrom or who is predicted to suffer from said disease or who has been diagnosed to potentially develop the disease), the method comprising administering to the subject a combination therapy comprising a therapeutically effective amount of GA and a therapeutically effective amount of CBD.

In some embodiments, the GA is administered by injection.

In further embodiments, the GA is administered by s.c. injection.

An important attribute of the presently conceived combination therapy is that it is effective for treating, preventing, ameliorating or delaying the onset of MS compared to each one of its components, GA or CBD, alone. The term ‘effective’ with respect to the presently proposed combination therapy applies to a number of situations: 1—when the GA and CBD combination is more effective for a reduction primary and/or secondary symptoms of MS (at least one symptom) than each one of the components alone; 2—when the combination is more effective for a reduction of GA or CBD adverse reactions (at least one symptom) when being administered alone; and 3—when the combination is effective for a reduction of GA or CBD therapeutic dose compared to GA or CBD being administered alone. The concept of being effective is further applied in the context of onset of a symptom, duration, severity, relapse and overall occurrence thereof.

Thus, in certain aspects, the invention can be articulated as a method for treating, preventing, ameliorating or delaying the onset of MS in a subject in a need thereof, said method comprises administrating to said subject a combination therapy comprising a therapeutically effective amount of injected GA and a therapeutically effective amount of CBD, wherein the combination therapy is effective for treating, preventing, ameliorating or delaying the onset of MS.

From another point of view, the invention can be articulated as an effective amount of injected GA for use in a method of administering to a subject an effective amount of CBD, wherein the effective amount of GA is selected to potentiate at least one effect associated with the effective amount of CBD. In this context, the term ‘potentiate’ and ‘effective’ herein are analogous, as defined herein and as known in the art.

The therapeutic concept using a combination therapy for MS, with its implied methods and compositions and kits, can be applied for treating a chronic MS or an acute MS, or for preventing development, deterioration or relapse of MS. In other words, the present therapies can be applied to a subject suffering from MS or a subject who is predicted to suffer from MS or who has been diagnosed to potentially develop the disease.

In this connection, a number of clinical tools have been developed for the prediction of MS predisposition, severity and relapse, such as examining vitamin D blood levels is used for the prediction of risk of developing MS, there are a number of immune markers for predicting severity and clinical course in MS, and more recently—genetic markers.

In some embodiments, the present combination therapy are applicable to the prevention of a relapse of MS, e.g., reducing the frequency of relapses in patients with relapsing-remitting multiple sclerosis (RRMS).

More specifically, MS as a clinical entity is highly heterogeneous with symptoms varying from person to person and over the course of time. Classical MS includes:
Numbness or weakness in one or more limbs, typically unilateral, legs and trunk;
Partial or complete loss of vision, usually in one eye at a time, often with pain during eye movement;
Prolonged double vision;
Tingling or pain in various body parts;
Electric-shock sensations that occur with certain neck movements (Lhermitte sign);
Tremor, lack of coordination or unsteady gait;
Slurred speech;
Fatigue;
Dizziness; and/or
Problems with bowel and bladder function.

In some embodiments, the combination therapy of the invention is effective for treating, preventing, ameliorating or delaying at least one symptom of MS.

As has been noted, most MS patients have a relapsing-remitting disease course (RRMS), certain patients with RRMS eventually develop a steady progression of symptoms, known as SPMS.

In some embodiments, the combination therapy according to the invention is effective for treating, preventing, ameliorating or delaying at least one symptom of MS relapse in RRMS, at least one symptom of SPMS.

In certain embodiments, the combination therapy is effective for treating, preventing, ameliorating or delaying problems with mobility and gait specifically associated SPMS.

In numerous embodiments, the terms ‘preventing’ and ‘delaying’ are used herein to convey a postponement in the onset and occurrence of the disease, avoidance of recurrence, or reduction of risk of a recurrence of MS episode, specifically in populations at risk, including among others patients with family history of MS, certain viral infections (most notably Epstein-Barr virus), certain autoimmune diseases (e.g. thyroid disease, type 1 diabetes and inflammatory bowel disease), smoking patients (smokers are more likely to develop RRMS).

The terms ‘preventing’ and ‘delaying’ are further used to convey a reduction of secondary symptoms related to MS condition, including (among others):
Muscle stiffness or spasms;
Paralysis, typically in the legs;
Problems with bladder, bowel or sexual function;
Mental changes, such as forgetfulness or mood swings;
Depression; and/or
Epilepsy.

It is further conceived that the present invention provides a method for reducing, inhibiting, attenuating or eliminating at least one side effect associated with administration of GA to a subject, said method comprising administering an effective amount of CBD to the patient, such that the effective amount is sufficient to reduce said side effect.

It is conceived that in numerous embodiments at least one side effect is associated with GA administered by injection. In some embodiments, the at least one side effect is associated with a subcutaneous injection of GA.

Common side effects associated with GA (Copaxone) include:
injection site reactions (e.g., pain, redness, soreness, itching, swelling, or lump);
nausea, vomiting;
chills, weakness, fever or flu symptoms;
joint aches, body aches, including neck pain, back, pain
double vision;
headache;
increased urge to urinate;
swelling in hands or feet;
vaginal itching or discharge; and/or
white patches or sores in the mouth or on your lips.

Immediate reactions can include:
flushing (warmth, redness, or tingly feeling);
chest pain;
fast heartbeat;
anxiety;
shortness of breath, and/or
itching,

Serious side effects include:
dizziness;
fainting;
infection (such as fever, persistent sore throat);
mental/mood changes (such as depression);
severe pain at the injection site;
shakiness (tremor); and/or
vision problems.

It is conceived that at least one of the above side effects is reduced in terms of onset, occurrence or severity due to the combined administration of an effective amount of pure or purified CBD in concurrence with the administration of GA.

It is further conceived that the subject to be treated with a combination therapy of the invention is one which has been found or determined to possibly benefit from the treatment with GA, and for whom a therapeutic protocol has been tailored, specifying effective amounts of the GA and the CBD, such that the amount of one component is adjusted to or determined based on the amount of the other component or to any one other protocol parameter which may, inter alia, depend on subject health and personal factors as well as on time of administration, sequence and administration regimen.

In other words, according to the invention the effective therapeutic doses of the CBD and GA components in the combined therapy are subject to personalized dosing regimens determined by the treating physician.

Many MS patients are treated with GA in the form of Copaxone using regimen of 20 mg administered in a single s.c. administration. Thus, in numerous embodiments daily doses of GA and CBD in the combination therapy of the invention are: 20 mg GA in the form of Copaxone administered in a single s.c. administration, and CBD in the range of 100-1500 mg in a formulation adapted for oral or s.c. or dermal administration.

In some embodiments, Copaxone is administered in the form of 20 mg in a single s.c. administration, and CBD in a daily dose of 1.00, 200, 300, 400, 500, 600, 700, 800, 900, 1000, 1100, 1200, 1300, 1400, 1500 mg, or more, to achieve the therapeutic effects referred to above.

The CBD can be administered before or after or simultaneously with the s.c. injection of Copaxone, in the form of oil, for example, for an oral or dermal (also s.c.) application.

MS patients treated with Copaxone also use 40 mg administered s.c. three times per week, and at least 48 hours apart. Thus, in further embodiments daily doses of the GA and CBD in the combination therapy of the invention are, for example: 40 mg GA in the form of Copaxone administered three times per week in at least 48 hours intervals, and CBD in the range of 100-1500 mg per day in a formulation adapted for oral or s.c. administration.

In such cases Copaxone is administered s.c. in the form of 40 mg three times per week (i.e., 120 mg weekly dose), and CBD may be administered as 100, 200, 300, 400, 500, 600, 700, 800, 900, 1000, 1100, 1200, 1300, 1400, 1500 mg per day (or 700, 1400, 2100, 2800, 3500, 4200.4900, 5600, 6300, 7000, 7700, $400, 9100, 9800, 10500 mg per week), or more, to achieve the therapeutic effects referred to above.

In such cases, the CBD may be administered together or apart, before or between Copaxone injections.

More specifically, the subject to be treated is one who, at the time of assessment, is treated with injectable GA, and the amount and frequency of administration of the CBD is determined in consideration of the concurrent GA treatment.

In some embodiments, the subject is one who is predisposed, suspected or known to suffer from injectable GA-related side effects, whereby the administration of the CBD, as defined herein, assists in reducing or diminishing such side effects.

In some embodiments, the CBD is administered prior to the administration of GA. In some embodiments, the CBD is administered immediately after administration of GA. In other embodiments, the two are administered simultaneously either in separate dosage forms or as a single mixed composition of matter comprising GA and CBD in a carrier suitable for injection, preferably for s.c. injection

In some embodiments, the at least one side effect is a skin lesion induced by s.c. administration of GA. As described herein, such skin lesion may be any lesion known in the art to result from local injection site reactions.

When referring to glatiramer acetate (GA, also known as Copolymer 1, Cop-1, the active ingredient of Copaxone as marketed by Teva Pharmaceuticals) is meant a random polymer consisting of acetate salts of synthetic polypeptides, containing four naturally occurring amino acids: L-glutamic acid, L-alanine, L-tyrosine, and L-lysine, with the average molecular weight of 4.7-13 KDa. GA is identified by specific antibodies, its biological activity is determined by its ability to block the induction of EAE in mice.

Under Copaxone is meant a composition known as such and available as a clear, colorless to slightly yellow, sterile, nonpyrogenic solution for s.c. injection wherein each 1 mL contains 20 mg or 40 mg GA and 40 mg mannitol in pH of about 5.5 to 7.0.

In a broader sense, the term ‘GA’ (Co-1) denotes a synthetic analogue of myelin basic protein (MBP) believed to be important in the process of myelination of nerves in the nervous system, and therefore implicated in the pathogenesis of MS. Its therapeutic effects in the treatment of MS are thought to be via immunomodulation and neuroprotection.

It is conceivable that GA analogues and derivatives acting by the same biological mechanism can be part of the same combination therapy. For example, Plovamer Acetate (also PA or Cop-2) is a structurally similar copolymer mixture of four amino acids of defined ratio, it has been rationally designed to have improved efficacy over GA. PA and GA share a similar mechanism of action, both competitively bind to MHC II and drive T-helper cell (Th)2-like responses. PA is undergoing Phase II testing for RIMS.

Thus, the term GA as used herein encompasses a variety of amino acid copolymers mimicking MBP with a common biological feature of alleviation of EAR

The effect of oral GA was tested in both rodents and primates in acute as well as in chronic/relapsing models of EAE. Oral GA was found to suppress acute EAE induced in rats, mice, and rhesus monkeys. Thus the term ‘GA’ as used herein does not necessarily related to an injected GA, or s.c. injected GA, but is further applicable to GA administered via other routes, including the oral route.

The terms cannabidiol or CBD, both denote the compound 2-[(1R,6R)-6-isopropenyl-3-methylcyclohex-2-en-1-yl]-5-pentylbenzene-1,3-diol or an enantiomers thereof. It further denotes a compound of the following formula:

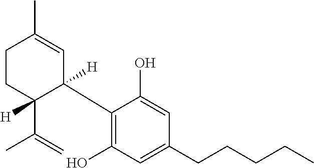 [SMILES: C=C(C)[C@@H]1CCC(C)=C[C@H]1c1c(O)cc(CCCCC)cc1O]

In this connection the term ‘cannabidiol’ applies to a synthetic or an isolated CBD. It should be emphasized that within the context of the present invention, this term does not apply to a cannabis, or a crude cannabis or hemp extract, or tinctures of plant material naturally containing CBD. A synthetic CBD can be produced by means of various methods, examples of such methods are described in PCT/IL01/00537 (U.S. Pat. No. 8,071,641). Pure or a substantially pure CBD can be obtained from a plant material by a number of methods, examples of those are described in U.S. Pat. No. 6,403,123 and Gaoni and Mechoulam (J. Am. Chem. Soc. 93: 217-224 (1971).

Under a pure or an isolated CBD is meant a preparation of CBD with chromatographic purity of 95% or greater, 96% or greater, 97% or greater, 98% or greater, or 99% or greater, or 99.5% or greater, as determined by HPLC, for example. Synthetic or purified CBD can be stored as in a crystalline form or as ethanolic solutions.

In some embodiments, the CBD refers to a formulation or a material consisting CBD, or a formulation or a material that is free of any other cannabis derived material. In some embodiments, the CBD is free of THC.

From a broader point of view, CBD is a non-psychotropic cannainoid which has a very low affinity for the cannabinoid CB1 and CB2 receptors, but acts as an indirect antagonist of these receptors. Therefore, the terms ‘synthetic’, ‘isolated’ or ‘purified CBD’ further encompass CBD enantiomers and derivatives displaying the same activity and the same mechanism of action. For example, cannabidiol-dimethylheptyl (also known as CBD-DMH or DMH-CBD) is a synthetic CBD homologue with a replacement of the pentyl chain for a dimethylheptyl chain. This compound is not psychoactive and has similar anticonvulsant and anti-inflammatory to CBD.

As described herein, it was found that a combination therapy comprising CBD and s.c injected GA has profound effects both therapeutically and in reducing side effects associated with injectable GA. It should be understood that reference to administration of GA with CBD in ‘combination’ or ‘together’ refers to a treatment schedule involving more than one type of therapy. In accordance with the present disclosure, combination therapy denotes a regimen involving administration of at least two substances in a single treatment cycle. As may be appreciated, the combination therapy requires an assessment of various parameters, inter alia, treatment schedule, predetermined ratio of the CBD and GA (dosing), number of treatment cycles, and others. Assessment of the combination therapy may be carried out before onset of treatment. For example, the CBD may be administered during the period at which a patient is treated with GA, at any time before administration of GA or at any time after administration of GA, provided that the later administration is at a time sufficient to yield an effective combination therapy.

The CBD and GA described herein can be administered and dosed by the methods of the invention, in accordance with good medical practice, such as systemically, for example by parenteral, e.g. intravenous, intraperitoneal or intramuscular injection. In another example, the CBD and GA can be introduced to a site by any suitable route including intravenous, subcutaneous, transcutaneous, topical, intramuscular, intraarticular, subconjunctival, or mucosal, e.g. oral, or intraocular administration. The administration of the two components may be by the same or different modes of administration and at the same or different frequency, i.e. at the same or different time points.

In accordance with some embodiments of the present disclosure, administration of GA and the CBD is at different dosage forms. In other words, GA and the CBD are not present in the same composition. As detailed above, various modes of administration are known in the art, including, but are not limiting to, topical administration, enteral administration, parental administration, oral administration, administration by inhalation. For example by injection (e.g., using a subcutaneous, intramuscular, intravenous, intraperitoneal, or intradermal injection), infusion (e.g. intraperitoneal), transdermal, transmucosal, by inhalation or sublingually.

For example, GA may be parenterally administered, preferably by s.c. administration and the CBD may be administered orally, sublingually, by injection (such as s.c.), by inhalation, by smoking intranasal transdermal or topical (topical administration being preferable at the site of injection). Preferably the two are not both administered nasally, most preferably none of the two are administered nasally.

In some embodiments, GA and CBD can be administered in different routes. In some preferred embodiments, GA is s.c. administrated. In some other embodiments, the CBD is administrated topically, orally, sublingually or by inhalation. In some further embodiments, the CBD is administered is by intraperitoneal administration. In some further embodiments, the CBD is administered by transdermal administration. In some further embodiments, the CBD is administrated by suppositories. As appreciated, CBD may be administered as drops, spray, or by nebulation. In some further embodiments, the CBD is sprayed into the mouth. In some further embodiments, GA is s.c. administrated and the CBD is administered by transdermal administration. In some further embodiments, GA is s.c. administrated and the CBD is administrated by suppositories. In some embodiments, the CBD is topically administrated, preferably at the site of injection. As described, GA may be injected s.c. into the abdomen, thigh (right and/or left), arm (right and/or left) and hip (right and/or left). In some embodiments, GA is administrated by s.c. injection into the abdomen and the CBD is administrated by i.p. injection or infusion or by any other way which would increase the combined effect and reduce side effects associated with GA administration.

In some embodiments, each of GA and the CBD is administered separately, i.e. not within the same composition/dosage form. Such an administration allows each component to be administered at a most effective site. In other words, GA may be administered s.c. and the CBD may be administered topically or orally or by inhalation.

In some embodiments, GA may be administered s.c. and the CBD may be administered topically. In some further embodiments, the CBD may be administered locally to the site of GA injection, preferably as a time window close to the injection (before or after).

In some embodiments, GA and the CBD may be each administrated on a daily basis. In some other embodiments, GA may be administered three time a week and the CBD may be administrated on a daily basis. For example, GA may be administered at a daily dose of 20 mg by s.c. injection or at a dose of 40 mg injected three times a week and the CBD either daily or also three times week on the same days the GA is administered. The CBD is administered daily or 3 times a week at a dose of 10 to 1500 mg a day, preferably 1-3 mg/kg/a day, or 10-150 mg a day, or 200, 300, 400 mg a day, or 1200 to 1500 mg a day.

In some embodiments, the GA and CBD are administered simultaneously, in distinct compositions or in the same composition, adapted for parenteral, administration, oral or transdermal administration (also including s.c.).

In some embodiments, especially when the GA and the CBD are present in the same composition, such compositions are adapted to be administered by s.c. injection.

Thus, in yet another aspect, the invention provides a pharmaceutical composition comprising GA and CBD in a carrier adapted for s.c. injection. Such compositions are intended for use in treating, preventing, ameliorating or delaying the onset of MS in a subject suffering therefrom or who is predicted to suffer from said disease or who has been diagnosed to potentially develop the disease.

In numerous embodiments the pharmaceutical composition can further comprise additional constitutes, excipients and/or drugs facilitating relevant to the symptoms, disease and administration route.

As detailed herein various modes of administration are known in the art to be applicable for administration of the composition comprising GA and CBD. For example, topical administration, enteral administration, parental administration. More specifically, the composition comprising GA and the CBD may be administered by injection (e.g., using a subcutaneous, intramuscular, intravenous, intraperitoneal, or intradermal injection), infusion (e.g. intraperitoneal), transdermal, transmucosal, orally, by suppository, by inhalation, or sublingually. In accordance with such embodiments, the composition comprising GA and the CBD is not administered by intranasal administration.

In some embodiments, the composition comprising GA and the CBD is administered by injection, e.g., by s.c. injection.

For injection, the active ingredients of the pharmaceutical composition may be formulated in organic solutions, preferably in oily carriers, such as arachis oil, sesame oil, olive oil and similar carriers, or liposome carriers, to which yet other excipients may, if desired, be added, such as benzyl alcohol and benzyl benzoate

One route of administration which is suited for the pharmaceutical compositions of the present invention is sub-periosteal injection, as described in U.S. Pat. No. 6,525,030 to Erikkson. For transmucosal administration, penetrants appropriate to the barrier to be permeated are used in the formulation. Such penetrants are generally known in the art.

The CBD and GA may each be administrated to a subject in need thereof in an effective amount. As known, the ‘effective amount’ for purposes herein may be determined by such considerations as known in the art. The amount must be effective to achieve the desired therapeutic effect, depending, inter alia, on the type and severity of the disease to be treated and the treatment regime. As generally known, the effective amount depends on a variety of factors including for example the distribution profile within the body, a variety of pharmacological parameters such as half-life in the body, on undesired side effects, if any, on factors such as age and gender, and others. In the context of the present disclosure and as described herein, the effective amount of the CBD and the effective amount of GA are selected such that the combination has the desired therapeutic effect. Typically, the GA is administered in doses of 20, 40, or 80 mg a day or 3 times a week and CBD in doses ranging from 10-1500 mg, preferably 10, 500, more preferably 10-150 mg a day or 3 times a week.

Another aspect of the invention is to provide use of GA for the preparation of a composition to be administered in combination with CBD.

Yet another aspect is to provide use of CBD for the preparation of a composition to be administered in combination with GA.

In some embodiments, the composition of GA or CBD is formulated separately from a composition of the CBD or composition comprising GA, respectively. In some further embodiments and as detailed herein, GA and the CBD are formulated within the same pharmaceutical composition, mostly in formulations adapted to carrier both lipophilic agents (CBD) and hydrophilic agents (GA) such as liposomes, micro and nano emulsions, nanocarriers and nano or microcapsules etc.

The invention further provides a pharmaceutical kit comprising in separate reservoirs an effective amount of GA and an effective amount CBD, and further comprising instructions for using said effective amount of GA and effective amount of CBD in a combination therapy for the treatment of MS.

In some embodiments, the different reservoirs are different syringes or different formulation containers comprising the actives in solid or liquid or solution forms or the CBD is in a form suitable for inhalation.

As has been noted, in the present disclosure, treating, preventing, ameliorating or delaying MS refers to achieving a state of absence of disease activity in patients known to have the disease or who are predisposed to having the disease or who have been diagnosed as having the disease. In connection with MS, it is commonly used to refer to absence of active MS when this disease is expected to manifest again in the future. As MS is associated with symptoms occurring either in discrete episodes (relapsing forms) or slowly accumulating over time (progressive forms), a partial remission may be defined as 50 percent or greater reduction in the intensity and frequency of episodes or attacks. A complete remission may be defined as complete disappearance of all such manifestations of disease.

In some embodiments, treatment of MS refers to reducing the frequency of relapses in patients with relapsing-remitting multiple sclerosis (RRMS). In combination of the invention, each of the two components used in the combination therapy may potentiate the other. The term ‘potentiate’ as used herein refers to a pharmacologic response (effect) that is a result of the combination therapy and which is greater than each of the individual responses to each component or agent. When referring to potentiating of a therapeutic effect of GA by the CBD it should be noted to encompasses at least one of the following (i) an improvement of at least one diagnostic parameter in the patient treated with the combination therapy compared to a patient treated with GA alone or CBD alone or (ii) obtaining the same improvement but with a lower GA dose or a longer doing interval.

The at least one diagnostic parameter may refer to any parameter used in diagnosis or prognosis of MS such as oligoclonal band. Oligoclonal bands (OCBs) are bands of immunoglobulins detected in a patient's blood serum, or cerebrospinal fluid (CSF). In MS, OCBs of immunoglobulin G antibodies are usually detected. In addition, at least one parameter may refer to any criteria known in the art for diagnosis or prognosis of MS such as Poser criteria or McDonald criteria. As appreciated, each one of these criteria refers a set of rules yielding conclusions related to the patient status. For example, the Poster criteria refers to a set of rules that can yield five conclusions: CDMS, LSDMS, CPMS, LSPMS or no MS, defined as follows: (1) CDMS—clinically definite MS: needs two attacks and some clinical or para-clinical evidences, (2) LSDMS—laboratory supported definite MS, showing oligoclonal bands and clinical or paraclinical evidences, (3) CPMS—clinically probable MS, with less restrict combinations, (4) LSPMS—Laboratory supported probable MS: only two attacks are enough to enter this category, and (5) no MS—there is no clinical evidence of having MS. The McDonald criteria maintained a scheme for diagnosing MS based on clinical grounds but also proposed that when clinical evidence is lacking, magnetic resonance imaging (MRI) findings can be used and this criteria may be used to facilitate the diagnosis of MS in patients who present with their first demyelinating attack and significantly increase the sensitivity for diagnosing MS without compromising the specificity.

The terms ‘treatment’ or ‘prevention’ herein refers to the complete range of therapeutically positive effects of administrating to a subject including inhibition, reduction of, alleviation of, and relief from, a condition as detailed herein. More specifically, treatment or prevention of relapse or recurrence of the disease include the prevention or postponement of development of the disease, prevention or postponement of development of symptoms and/or a reduction in the severity of such symptoms that will or are expected to develop. These further include ameliorating existing symptoms, preventing additional symptoms, and ameliorating or preventing the underlying metabolic causes of symptoms.

It should be appreciated that the terms ‘inhibition’, ‘reduction’, ‘attenuation’ as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

As described herein, s.c. administration of GA may result in occurrence of skin lesion. For example, a permanent indentation under the skin (lipoatrophy or, rarely, necrosis) at the injection site may occur, due to local destruction of fat tissue. This is also indicated by the recommendation of GA use, namely to follow proper injection technique and monitor any skin changes. The inventor has surprisingly found that when GA was injected s.c in combination with the CBD, an elimination or reduction of these skin lesions were observed.

A skin lesion or injection site reaction as described herein refers to local skin reaction that occur upon administration of GA. Such lesion or reaction occurs usually when the drug escapes into the skin, resulting in different appearance compared to the skin around it. Exemplary skin lesion or site reaction include a scar, edema, pain, redness, soreness, and itching, swelling, or hard lump. The skin lesion described herein is a benign skin lesion and as described herein, can be characterized by histology. As such, the combination therapy is particularly suitable for administration to patients who are suffering from a skin lesion following treatment with GA. In the context of the present disclosure, a reduction in a skin lesion refers to at least one of a reduction in the severity of a skin lesion, reduction in the frequency of appearance of a skin lesion, reduction in the number of skin lesions or prevention of a skin lesion.

### NON-LIMITING EXAMPLES

### Example 1: Suppression of EAE in SJL Mice by CBD with or without GA Materials

CBD was obtained from THC Pharm GmBh (Germany).
Proteolipid protein, (PLP) (139-151) were purchased from (GP, China)
CFA and Pertusis toxin (PT) were purchased from Sigma.

### Methods

SJL/J female mice were purchased from Harlen. Mice at the age of 6-7 weeks old were used to the in the experiments described below. All the experiments were done in accordance with the protocol approved by the Ethics Committee of the Hebrew University of Jerusalem. The mice were housed in cages with free access to food and water. They were maintained in 12 hr. light/dark cycle at room temperature.

Mice were immunized with PLP (139-151) emulsified in CFA together with pertussis vaccine according to the method described in Hooke Laboratories protocols (http://hookelabs.com/protocols/eaeAISJL). PLP is used to induce relapsing-remitting (RR)-EAE model. In the EAE experiments, SJL/J female mice at an age of 6-7 weeks old were used. Mice were observed daily for the appearance of neurological paralytic symptoms (6-7 mice/group), and were scored in scale from 0-5 denoting as follows:

| | |
|---|---|
| 0 | no neurological sign |
| 0.5 | distal limb |
| 1 | limp fail |
| 2 | loose of righting reflex (difficulty to turnover when laid in the back) |
| 3 | ataxin hind limp paralysis (hind limbs are dragged) |
| 4 | paralysis of the hind legs |
| 5 | full paralysis (immobility) |
| 6 | death of the mice |

### Statistical Evaluation

Raw p-value were obtained from exact one-tail Mann Whitney tests and adjusted for multiple comparisons (within each experiment) using Holm modification of the Bonferroni correction

Histology Following s.c. injection of PLP (139-151) mixed with CFA as well as two pertussis-toxin injections and appearance of paralysis signs in the mice (usually after 9-11 days after initiation of EAE) treatment of mice began.

The treatments were given daily, five times a week and usually continued for about 60 days. GA were given s.c. (at an injection dose of 1 mg/mouse), and CBD at a dose of 5 mg/kg were administrated i.p. (intraperitoneal).

Administration of PLP 139-151 induced 3 phases of paralysis and in one experiment it induced only one phase. As known, 3 peaks are considered more similar to physiological manifestations in humans, however, one peak is considered acceptable and is used in many MS studies.

### Results

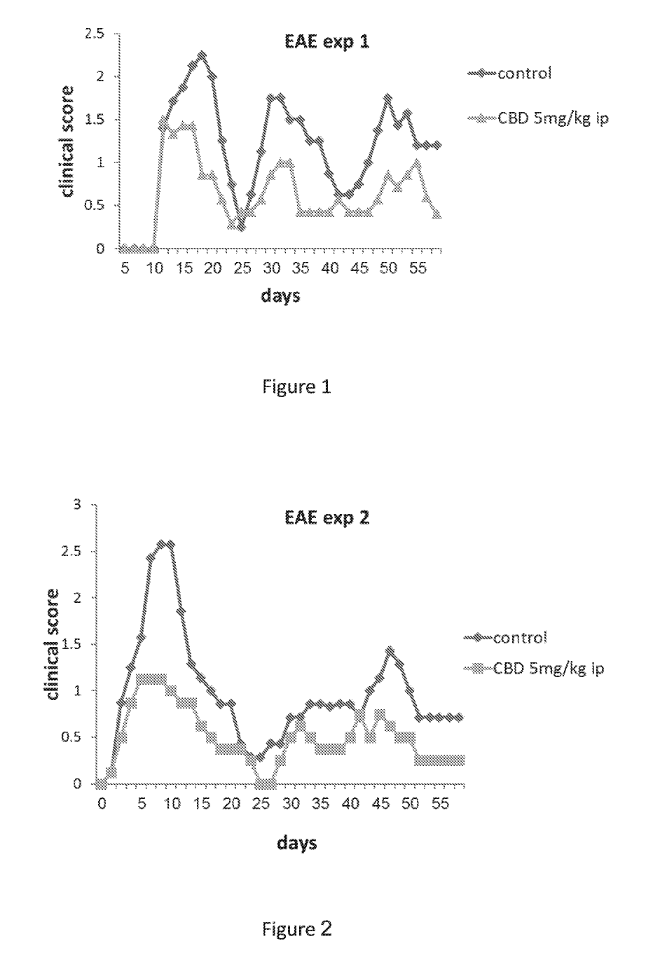

Effects of CBD monotherapy were demonstrated in the EAE model in mice. FIG. 1 shows an EAE with 3 pronounced relapse emission cycles, and FIG. 2 with less pronounces cycles. In both cases CBD 5 mf/kg injected ip caused a significant decrease in clinical score

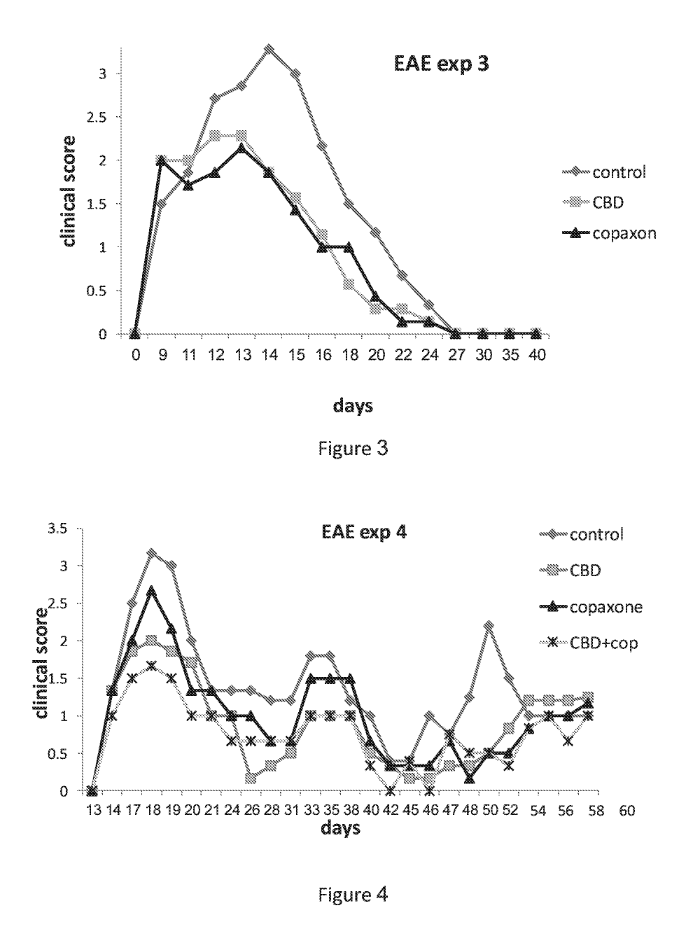

Comparative effects of CBD and Copaxone monotherapies were further studied in the same model. FIG. 3 shows data in mice administered with CBD 5 mg/kg (administered ip) or Copaxone (1 mg/mice injected s.c.). Treatment with the CBD alone or Copaxone alone significantly decreased clinical score.

The effect of the combination therapy of the invention was demonstrated in the same experimental model. FIG. 4 shows a longitudinal study using 1-3 cycles of the mono- or combination therapies administered as in FIG. 3 above. Data shows the effect the combination therapy comprising of CBD and Copaxone (i.e., GA) was significantly greater in terms of clinical scores than the effect of each one of the monotherapies alone.

### Pathology

Studies of monotherapies (CBD or Copaxone) and the combination therapy (CBD plus Copaxone) also included histological studies of infiltration of immune cells into the spinal cord in EAE mice (i.e., MS core pathology). Sixty days following injection of PLP into control non-treated SJL female mice, a massive infiltration of immune cells into the white section of the spinal cord was observed in eosin/hematoxylin staining sections. In contrast, an almost total reduction of such infiltration into the spinal cord was observed following treatments with either CBD, Copaxone or Copaxone plus CBD.

### Cutaneous Wound

Specific effects of the combination therapy (CBD plus Copaxone) were observed at the site of subcutaneous Copaxone injection. In mice treated with the Copaxone only (2 out of 7 mice), a skin lesion developed on the back at the site of repeated Copaxone injections. No suchlesions were observed in mice treated with CBD or with the CBD plus Copaxone combination therapy. The lesion (dermal wound) was characterized by skin swelling, open wound that did not heal and the appearance of blood clot.

These results demonstrated that co-administration of Copaxone with CBD prevented the formation of cutaneous wound characteristic of Copaxone when s.c. administered alone.

1.-45. (canceled)

46. A method for reducing, inhibiting, attenuating or eliminating at least one side effect associated with injecting glatiramer acetate (GA), said method comprising administering to a subject an oral composition comprising an effective amount of cannabidiol (CBD) free of any other cannabinoids.

47. The method according to claim 46, wherein said injecting GA is administered for treating, preventing, ameliorating or delaying the onset of multiple sclerosis (MS).

48. The method according to claim 46, wherein the at least one side effect associated with injecting GA is an injection site reaction.

49. The method according to claim 48, wherein the injection site reaction is selected from a scar, an edema, pain, redness, soreness, itching, swelling and hard lump.

50. The method according to claim 46, wherein the administering of the oral CBD composition is simultaneous or sequential with the GA injection, before or after the GA injection.

51. A method for treating, preventing, ameliorating or delaying the onset of multiple sclerosis (MS) in a subject, said method consists of a combination therapy comprising administering to the subject an effective amount of injected glatiramer acetate (GA) and an oral composition comprising an effective amount of cannabidiol (CBD) free of any other cannabinoids.

52. The method according to claim 51, wherein said treating, preventing, ameliorating or delaying the onset of MS further comprises reducing the frequency of relapses in patients with RRMS.

53. The method according to claim 51, wherein said combination therapy comprises simultaneous or sequential administering of the CBD composition with the GA injection, before or after the GA injection.

54. The method according claim 51, wherein the effective amount of CBD comprised in the composition is sufficient to reduce, inhibit, attenuate or eliminate at least one side effect associated with GA injection.

55. A method of potentiating therapeutic effect of CBD on MS, the CBD is comprised in an effective amount in an oral composition free of any other cannabinoids, said method comprise administering to a subject an effective amount of injected GA such that said effective amount of GA is selected to potentiate at least one effect related to the oral administering of CBD on treating, preventing, ameliorating or delaying the onset of MS or reducing the frequency of relapses in patients with RRMS.

56. The method according to claim 55, wherein the injected GA is administrated simultaneously or sequentially with the oral CBD composition, before or after the CBD composition.

57. A method of potentiating therapeutic effect of injected GA on MS, the method comprising administering to a subject an oral composition comprising an effective amount of CBD free of any other cannabinoids such that the effective amount of the CBD is selected to potentiate at least one effect related to the injected GA on treating, preventing, ameliorating or delaying the onset of MS or reducing the frequency of relapses in patients with RRMS.

58. The method according to claim 57, wherein the potentiating a therapeutic effect of GA on MS further comprises reducing or diminishing at least one side effect related to the GA injection, optionally the at least side being an injection site reaction.

59. The method according to claim 57, wherein the oral CBD composition is administered simultaneously or sequentially with the GA injection, before or after the GA injection.

60. A kit comprising an effective amount of injectable GA and an effective amount of CBD formulated for oral administering, the kit further comprising instructions for use of the GA and the CBD, in combination, for treating MS and/or reducing at least one side effect related to the GA injection.

61. The method according to claim 47, wherein the administering of the oral CBD composition is simultaneous or sequential with the GA injection, before or after the GA injection.

62. The method according to claim 49, wherein the administering of the oral CBD composition is simultaneous or sequential with the GA injection, before or after the GA injection.

63. The method according to claim 52, wherein said combination therapy comprises simultaneous or sequential administering of the CBD composition with the GA injection, before or after the GA injection.

64. The method according claim 52, wherein the effective amount of CBD comprised in the composition is sufficient to reduce, inhibit, attenuate or eliminate at least one side effect associated with GA injection.

65. The method according claim 53, wherein the effective amount of CBD comprised in the composition is sufficient to reduce, inhibit, attenuate or eliminate at least one side effect associated with GA injection.
